(19) United States
(12) Patent Application Publication
(10) Pub. No.: US 2026/0351019 A1
(43) Pub. Date: Nov. 5, 2026

(54) SYSTEMS AND METHODS FOR MINERAL MAPPING POROUS MEDIA

(71) Applicant: BP Corporation North America Inc., Houston, TX (US)

(72) Inventors: Jeremy T. First, Houston, TX (US); Yuliana Maria Zapata Arboleda, Katy, TX (US); Srivatsa Mudumba-Ramana, Reading (GB); Glen Gettemy, Katy, TX (US)

(73) Assignee: BP Corporation North America Inc., Houston, TX (US)

(21) Appl. No.: 19/662,963

(22) Filed: Apr. 29, 2026

Related U.S. Application Data
(60) Provisional application No. 63/797,754, filed on Apr. 30, 2025.

(51) Int. Cl. G06V 20/60; G01N 23/04; G01N 23/083; G06V 10/26; G06V 10/762; G06V 10/764; G06V 20/70

(52) U.S. Cl. CPC G06V 20/60; G01N 23/04; G01N 23/083; G06V 10/26; G06V 10/762; G06V 10/764; G01N 2223/401; G01N 2223/616; G06V 20/70

(57) ABSTRACT

A method includes receiving an image volume of a porous media sample, segmenting the image volume into a plurality of voxels including a plurality of solid voxels each associated with a solid phase of the porous media sample and a plurality of pore phase voxels each associated with a pore phase of the porous media sample, partitioning the plurality of solid voxels into a plurality of distinct grains of the porous media sample, assigning an X-ray attenuation value to each solid voxel of each of the plurality of grains with an average X-ray attenuation for the grain to provide a grain-averaged image volume of the porous media sample, and dividing the plurality of grains of the grain-averaged image volume into a plurality of distinct mineral types based on the X-ray attenuation values of the solid voxels of the plurality of grains to produce a mineral-classified image volume.

### CROSS-REFERENCE TO RELATED APPLICATIONS

The present application is a non-provisional patent application which claims benefit of U.S. provisional application No. 63/797,754, filed Apr. 30, 2025, entitled “Systems and Methods for Mineral Mapping Porous Media”, which is hereby incorporated herein in its entirety for all purposes.

### STATEMENT REGARDING FEDERALLY SPONSORED

### Research or Development

Not applicable.

### BACKGROUND

Micro computed tomography (µCT) may be performed on samples of porous media to generate a three dimensional (3D) image volume of the porous media for further analysis. For instance, such digital image volumes may be obtained from rock samples of a subterranean formation of interest to further understand and characterize the subterranean formation, particularly with respect to the ability of the subterranean formation to contain materials of interest such as hydrocarbons and the like. Numerical analysis of the image volume can be particularly useful in examining properties and pore-scale processes of the porous media sample such as, for example, fluid flow and transport properties, geomechanical properties, and electrical properties of the porous media sample.

### BRIEF SUMMARY OF THE DISCLOSURE

An embodiment of a computer-implemented method for mineral mapping an image volume representing a porous media sample comprises (a) receiving an image volume of a porous media sample, (b) segmenting the image volume into a plurality of voxels including a plurality of solid voxels each associated with a solid phase of the porous media sample and a plurality of pore phase voxels each associated with a pore phase of the porous media sample, (c) partitioning the plurality of solid voxels into a plurality of distinct grains of the porous media sample, (d) assigning an X-ray attenuation value to each solid voxel of each of the plurality of grains with an average X-ray attenuation for the grain to provide a grain-averaged image volume of the porous media sample, and (e) dividing the plurality of grains of the grain-averaged image volume into a plurality of distinct mineral types based on the X-ray attenuation values of the solid voxels of the plurality of grains to produce a mineral-classified image volume. In some embodiments, the method comprises (f) generating by a single source micro computed tomography (µCT) scanner the image volume of the porous media sample. In some embodiments, (b) comprises segmenting the image volume into the plurality of voxels including the plurality of solid voxels, the plurality of pore phase voxels, and a plurality of micro-porous volume (MPV) voxels. In certain embodiments, (e) comprises applying a clustering algorithm to the grain-averaged image volume to identify a plurality of discrete clusters in attenuation value for the plurality of grains. In certain embodiments, the clustering algorithm comprises a K-means clustering algorithm. In some embodiments, the method comprises (f) receiving a user-defined initial number of clusters for the plurality of mineral types. In some embodiments, (c) comprises applying a watershedding algorithm to the image volume. In certain embodiments, the method comprises (f) correlating each of the plurality of mineral types with a mineral corresponding to the mineral type to estimate a composition of the porous media sample. In certain embodiments, the estimated composition of the porous media sample is based on a known composition of an analogous porous media sample. In some embodiments, the method comprises (g) determining one or more rock properties or one or more transport properties of the porous media sample using the mineral-classified image volume and the estimated composition of the porous media sample.

An embodiment of a computer-implemented method for mineral mapping an image volume representing a porous media sample comprises (a) receiving an image volume of a porous media sample, (b) segmenting the image volume into a plurality of voxels including a plurality of solid voxels each associated with a solid phase of the porous media sample and a plurality of pore phase voxels each associated with a pore phase of the porous media sample, (c) partitioning the plurality of solid voxels into a plurality of distinct grains of the porous media sample, (d) reducing a variance of X-ray attenuation values for each solid voxel present in each of the plurality of grains to provide a smoothed image volume of the porous media sample, and (e) dividing the plurality of grains of the smoothed image volume into a plurality of distinct mineral types based on the X-ray attenuation values of the solid voxels of the plurality of grains to produce a mineral-classified image volume. In some embodiments, the method comprises (f) generating by a single source micro computed tomography (µCT) scanner the image volume of the porous media sample. In certain embodiments, (d) comprises replacing original X-ray attenuation values for each solid voxel present in each of the plurality of grains based on a statistical parameter of the original X-ray values of the solid voxels of the grain. In certain embodiments, the statistical parameter comprises an average X-ray attenuation value for the solid voxels of the grain. In some embodiments, (c) comprises applying a watershedding algorithm to the image volume. In some embodiments, the method comprises (f) correlating each of the plurality of mineral types with a mineral corresponding to the mineral type to estimate a composition of the porous media sample. In certain embodiments, the estimated composition of the porous media sample is based on a known composition of an analogous porous media sample. In certain embodiments, the method comprises (g) determining one or more rock properties or one or more transport properties of the porous media sample using the mineral-classified image volume and the estimated composition of the porous media sample.

An embodiment of a system for mineral mapping an image volume representing a porous media sample comprises a processor, and a memory coupled to the processor, wherein machine-readable instructions are stored in the memory, and wherein the machine-readable instructions, when executed on the processor, configure the processor to (a) receive an image volume of a porous media sample, (b) segment the image volume into a plurality of voxels including a plurality of solid voxels each associated with a solid phase of the porous media sample and a plurality of pore phase voxels each associated with a pore phase of the porous media sample, (c) partition the plurality of solid voxels into a plurality of distinct grains of the porous media sample, (d) assign an X-ray attenuation value to each solid voxel of each of the plurality of grains with an average X-ray attenuation for the grain to provide a grain-averaged image volume of the porous media sample, and (e) divide the plurality of grains of the grain-averaged image volume into a plurality of distinct mineral types based on the X-ray attenuation values of the solid voxels of the plurality of grains to produce a mineral-classified image volume. In some embodiments, the machine-readable instructions, when executed on the processor, configure the processor to (f) correlate each of the plurality of mineral types with a mineral corresponding to the mineral type to estimate a composition of the porous media sample.

Embodiments described herein comprise a combination of features and characteristics intended to address various shortcomings associated with certain prior devices, systems, and methods. The foregoing has outlined rather broadly the features and technical characteristics of the disclosed embodiments in order that the detailed description that follows may be better understood. The various characteristics and features described above, as well as others, will be readily apparent to those skilled in the art upon reading the following detailed description, and by referring to the accompanying drawings. It should be appreciated that the conception and the specific embodiments disclosed may be readily utilized as a basis for modifying or designing other structures for carrying out the same purposes as the disclosed embodiments. It should also be realized that such equivalent constructions do not depart from the spirit and scope of the principles disclosed herein.

### BRIEF DESCRIPTION OF THE DRAWINGS

For a detailed description of various exemplary embodiments, reference will now be made to the accompanying drawings in which:

FIG. 1 is a flowchart of a method for mineral mapping a porous media sample according to some embodiments;

FIG. 2 is a schematic view of exemplary onshore and offshore sources of rock samples and fluid sampling for analysis by embodiments of testing systems and methods according to some embodiments;

FIG. 3 is a schematic view of an embodiment of a testing system for analyzing rock samples according to some embodiments;

FIG. 4 is an exemplary original image slice of an image volume obtained from a porous media sample according to some embodiments;

FIG. 5 is an exemplary segmented image slice of an image volume obtained from a porous media sample according to some embodiments;

FIG. 6 is an exemplary grain partitioned image slice of an image volume obtained from a porous media sample according to some embodiments;

FIG. 7 is an exemplary grain-averaged image slice of an image volume obtained from a porous media sample according to some embodiments;

FIG. 8 is an exemplary fully-classified image slice of an image volume obtained from a porous media sample according to some embodiments;

FIG. 9 is a histogram of voxel X-ray attenuation values of an image volume according to some embodiments;

FIG. 10 is a block diagram of a computer system according to some embodiments; and

FIGS. 11 and 12 are flowcharts of methods for mineral mapping an image volume representing a porous media sample according to some embodiments.

### DETAILED DESCRIPTION

The following discussion is directed to various exemplary embodiments. However, one skilled in the art will understand that the examples disclosed herein have broad application, and that the discussion of any embodiment is meant only to be exemplary of that embodiment and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form, and some details of conventional elements may not be shown in the interest of clarity and conciseness.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

In the following discussion and in the claims, the terms "including" and "comprising" are used in an open-ended fashion, and thus should be interpreted to mean "including, but not limited to…” Also, the term "couple" or "couples" is intended to mean either an indirect or direct connection. Thus, if a first device couples to a second device, that connection may be through a direct connection, or through an indirect connection via other devices, components, and connections. In addition, as used herein, the terms "axial" and "axially" generally mean along or parallel to a central axis (e.g., central axis of a body or a port), while the terms "radial" and "radially" generally mean perpendicular to the central axis. For instance, an axial distance refers to a distance measured along or parallel to the central axis, and a radial distance means a distance measured perpendicular to the central axis.

As previously described, digital image volumes obtained through the performance of µCT on porous media samples may be particularly useful for characterizing the porous media sample including rock properties and pore-scale processes thereof, particularly when complimented by suitable numerical analysis of the image volume. Generally, µCT involves generating x-rays using a suitable x-ray source that pass through the porous media sample to thereby scan the porous media sample whereby a digital image volume is generated comprising a plurality of separate elements called volume elements, or more commonly “voxels”. For example, each voxel may be cubic, having a side of equal length in the orthogonal X, Y, and Z directions. µCT has a greater spatial resolution that is significantly greater than that of conventional tomography, where the individual voxels of the image volume generated from µCT having a size (e.g., a length, width, and/or height) of between approximately 1-100 microns. Such spatial resolution allows for the examination of pore-scale properties of the porous media sample that would not be visible when using conventional tomography. For instance, µCT may be used to map the pore phase throughout the porous media sample which may not be possible with conventional tomography.

Additionally, while generally providing a sufficient spatial resolution for examining porous media samples, µCT remains a practical solution in many applications. As an example, µCT is substantially less cumbersome, time consuming, and expensive as other processes such as scanning electron microscopy (SEM) which, while having spatial resolutions that may far exceed µCT, is generally much more difficult and resource intensive to implement than µCT, limiting its practical applicability relative to the more convenient, less resource intensive µCT. Moreover, µCT typically provides spatial resolution and other properties sufficient for the examination of porous media samples in many applications including, for example, the characterization of subterranean formations.

More specifically, the µCT often employed in analyzing porous media samples is often “single source” or “single energy” µCT which, in the interest of maximizing the applicability (e.g., ease, cost, and time required for implementation) of the µCT, utilizes a single X-ray source providing a single X-ray energy (e.g., often between 80 kilovolts (kV) to 160 kV) that is passed through the porous media sample to generate the image volume thereof. As the X-ray passes through the porous media sample, the X-ray is attenuated with the degree of attenuation contingent on the type of material through which it passes (e.g., pore phase, grain phase of different mineral types). The single image volume of the porous media sample may be obtained by, for example, rotating the single X-ray source around the porous media sample to acquire a set of two dimensional (2D) projections each encoding the cumulative effect of attenuation along the X-ray paths. These 2D projections may then be reconstructed via a tomographic reconstruction technique (e.g., filtered back-projection techniques, iterative reconstruction techniques) into a 3D image volume of the porous media sample.

The image volume generated by single source µCT contains a plurality of separate voxels each having an intensity that corresponds to an attenuation value of the given voxel. The attenuation value of a selected voxel may correspond to or correlate with a density of the material inhabiting the selected voxel. For example, voxels comprising materials that absorb relatively more X-rays (e.g., minerals of the porous media sample) may appear brighter in the image volume whereas voxels comprising materials that absorb fewer X-rays (e.g., void space) may appear darker in the image volume. Thus, each voxel of the image volume generated using single source µCT has a single value ascribed to it which is associated with a single attenuation value for the given voxel.

While single source µCT is a valuable tool for characterizing and analyzing porous materials, particularly with respect to identifying or mapping pore phase, grain phase, and micro-porous volume (MPV) (pore-structures that are smaller than the resolution of the image and thus cannot be resolved), single source µCT is limited in its ability to distinguish between different mineral types of the grains within a given porous media sample. In some embodiments, MPV may correspond to a specific mineral type such as clay and the like. The process of mapping different mineral types (e.g., having different chemical compositions) of the grains in an image volume is referred to herein as “mineral mapping” of the image volume. Particularly, single source µCT, when employing computationally efficient segmentation techniques such as clustering techniques and the like, has difficulty in distinguishing between different mineral types given that the attenuation values for different mineral types tend to “blur” together more than the attenuation values of grain phase versus pore phase, making it difficult for single source µCT to perform mineral mapping of image volumes with acceptable accuracy and precision.

Conventionally, the limitations of single source µCT with respect to mineral mapping have been addressed using more exotic and resource intensive hardware and/or software solutions. For instance, one approach to addressing this problem has been employing so called “dual source” or “dual energy” µCT in which a plurality of different X-ray sources and/or different X-ray energies are passed through the porous media sample, whereby an image volume may be generated in which each voxel has a plurality of separate attenuation values corresponding to the different X-ray sources/X-ray energies used to generate the image volume. For instance, a relatively high energy X-ray (e.g., above 100 kV), where Compton scattering is dominant, may be passed through the porous media sample along with a relatively low energy X-ray (e.g., less than 100 kV), where photoelectric absorption is dominant, to obtain information on a voxel-by-voxel basis corresponding to both density and atomic number. In this manner, by comparing the different attenuation values for each voxel, mineral mapping may be performed with greater accuracy and precision than with single source µCT.

However, dual source µCT is more resource intensive, requiring additional equipment and computation relative to single source µCT, limiting its practical applicability relative to single source µCT. As an alternative to dual source µCT, another hardware-intensive conventional solution involves using SEM combined with, for instance, quantitative evaluation of materials by scanning electron microscopy (QEMSCAN) to perform mineral mapping of porous media samples. However, such techniques have similar or greater limitations to their practical applicability as dual source µCT.

Further, another conventional approach to address the limitations of single source µCT without resorting to more expensive and resource intensive hardware is to employ more sophisticated analysis of the image volume generated by single source µCT. For example, artificial intelligence (AI) algorithms such as neural nets (NNs) may be employed to perform mineral mapping of image volumes generated by single source µCT to enhance the accuracy and precision thereof. However, such AI algorithms must be initially trained on a large volume of training data before they may be deployed for the purposes of mineral mapping, where such large volumes of training data (e.g., different porous media samples that have been previously mineral mapped) may be unavailable in many applications, limiting the practical applicability of AI algorithms in addressing this shortcoming of single source µCT. Moreover, such algorithms are substantially more computationally intensive than clustering techniques conventionally used to segment image volumes generated by single source µCT.

Accordingly, embodiments disclosed herein include methods and systems for mineral mapping a porous media sample using computationally efficient algorithms such as clustering algorithms and the like. Particularly, embodiments disclosed herein include segmenting an image volume into a plurality of grain phase voxels and pore phase voxels to map the solid matter (e.g., defined by a plurality of packed grains) and pore phase within the porous media sample. Additionally, embodiments disclosed herein include partitioning the image volume to divide the plurality of grain phase voxels into a plurality of distinct and separate grains of the image volume. For example, watershedding algorithms may be used to partition the grains of the image volume, as will be discussed further herein. Embodiments disclosed herein include smoothing the X-ray attenuation values for the grain phase voxels defining each grain of the image volume. In this manner, the different grains may be more easily divided into different classes using, for example, a clustering algorithm, avoiding the need to employ more exotic hardware and/or AI algorithms in order to identify different mineral types within the image volume.

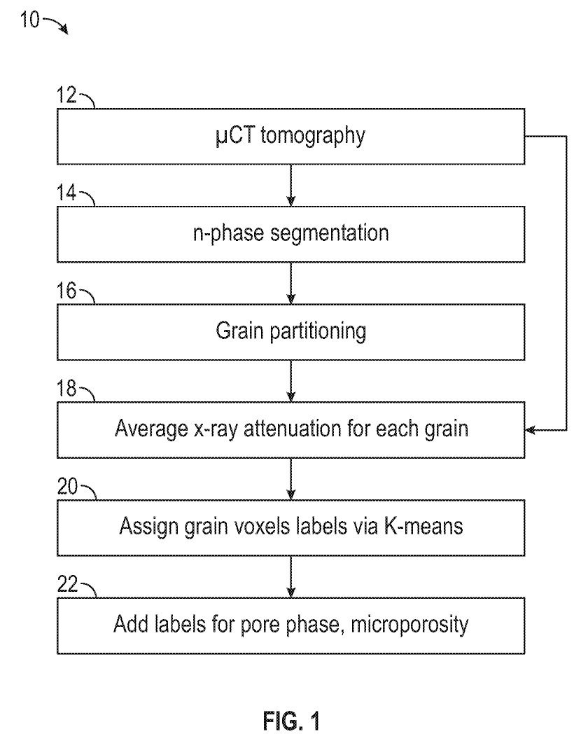

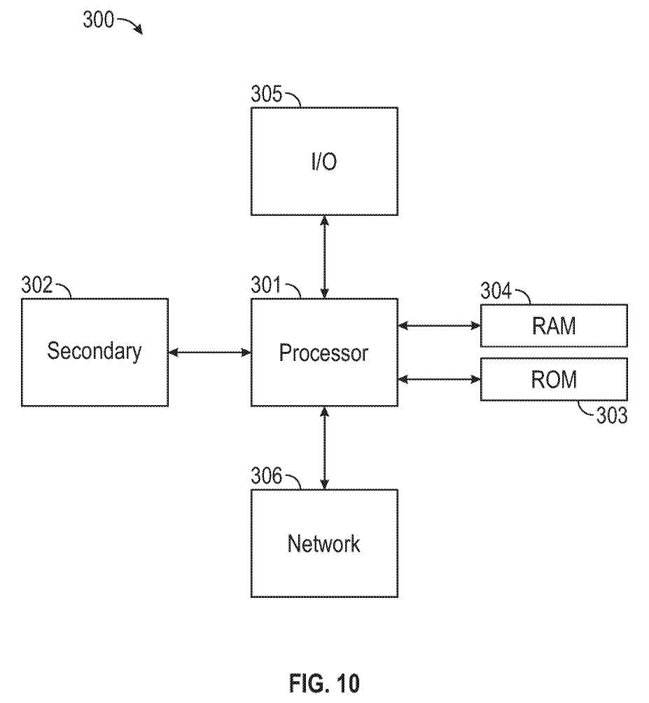

Referring to FIG. 1, a method 10 for mineral mapping a porous media sample according to some embodiments is shown. In this exemplary embodiment, one or more features of method 10 may be performed by, with, or using a computer system or device (or multiple computer devices). The computer device (or devices) may comprise or be similar to the example computer system 300 shown in FIG. 10 and described below.

Initially, at block 12 method 10 comprises performing µCT on a porous media sample such as, for instance, a rock from a subterranean formation. For instance, the digital image may comprise a digital image generated from a natural or synthetic rock sample, or other porous media. The rock sample (e.g., a natural rock sample) may comprise a rock sample from a subterranean formation of interest. Particularly, in this exemplary embodiment, the µCT performed on the porous media sample is a single source µCT in which a single X-ray source providing a single X-ray energy is passed through the porous media sample with the resulting image volume having a plurality of voxels each defined by a single attenuation value. Further, in some embodiments, rather than performing a µCT scan at block 12, block 12 of method 10 may instead include receiving a digital image of a porous media sample (e.g., a rock from a subterranean formation) that was obtained via a previously performed µCT scan of said porous media sample. Again, in at least some embodiments, the µCT used to generate the image volume received at block 12 is a single source µCT.

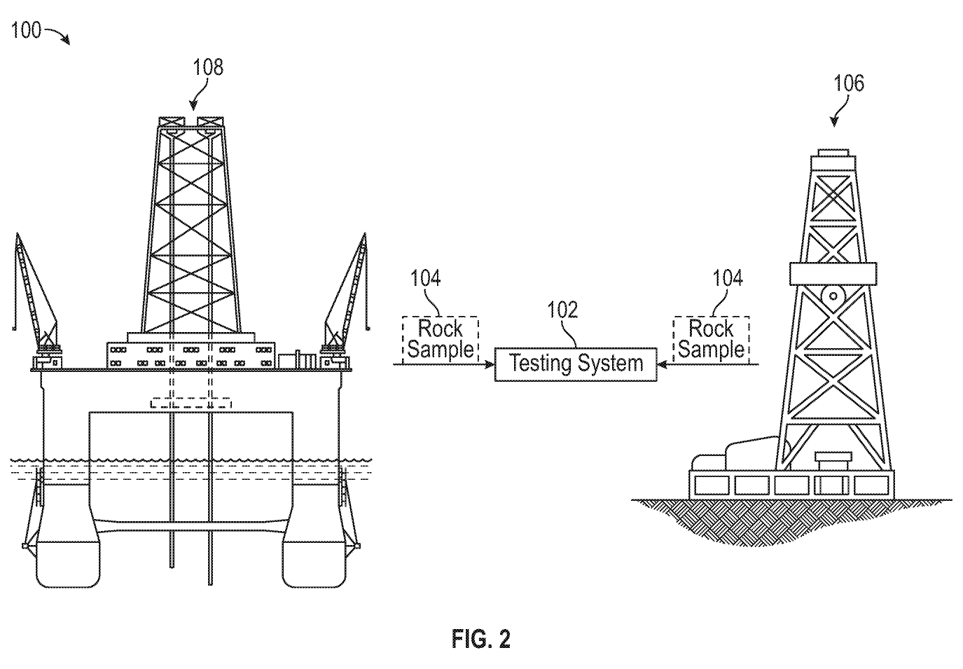

To provide an example, and referring to FIG. 2, a system 100 for acquiring and analyzing rock samples 104 for purposes of generating digital images of rock samples (e.g., the digital image generated or received at block 12 of method 10 in FIG. 1) is shown according to some embodiments. In some embodiments, system 100 may be used to analyze rock samples 104 that are obtained from a subterranean formation for purposes of enhancing or facilitating oil and gas production from the subterranean formation. For example, in some embodiments rock samples 104 can be obtained from terrestrial drilling system 106 or from marine (ocean, sea, lake, etc.) drilling system 108, either of which is utilized to extract resources such as hydrocarbons (oil, natural gas, etc.), water, and the like. In other embodiments, the subterranean formation may be used for different purposes such as, for example, geothermal energy applications, and the injection and storage of materials within the subterranean formation such as carbon capture and storage (CCS) and the like.

The way rock samples 104 are obtained and the physical form of those samples can vary widely. Examples of rock samples 104 useful in connection with embodiments disclosed herein include whole core samples, sidewall core samples (e.g., obtained from the sidewall of a wellbore extending through the subterranean formation), outcrop samples, drill cuttings, and laboratory generated synthetic rock samples such as sand packs and cemented packs.

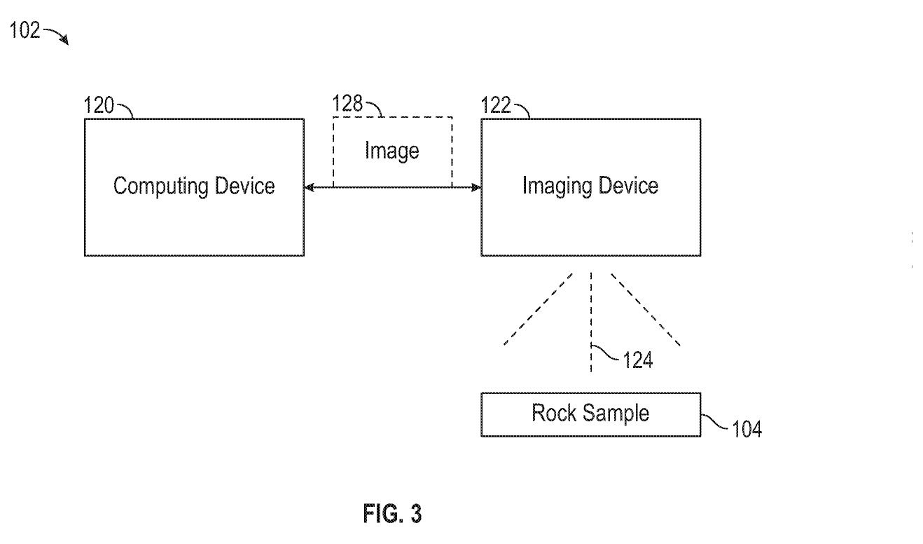

A testing system 102 of system 100 is configured to acquire and analyze image volumes 128 (shown in FIG. 3) of rock samples 104 in order to determine the physical properties of the corresponding sub-surface rock. The physical properties include, for example, petrophysical properties such as those petrophysical properties that are analyzed in the context of oil and gas exploration and production.

Referring to FIG. 3, a schematic diagram of testing system 102 is shown according to some embodiments. Testing system 102 includes imaging device 122 for obtaining 2D or 3D images, as well as other representations, of rock samples 104, such images and representations including details of the internal structure of the rock samples 104. An example of imaging device 122 is an X-ray CT scanner (or more simply “CT scanner”), having an X-ray source which emits X-ray radiation 124 that interacts with an object and measures the attenuation of that X-ray radiation 124 by the object in order to generate an image of its interior structure and constituents. The type, construction, or other attributes of imaging device 122 can correspond to that of any type of X-ray device, such as a micro-CT scanner, capable of producing an image representative of the internal structure of rock sample 104. In some embodiments, imaging device 122 comprises a single source µCT scanner having a single X-ray source that provides a single X-ray energy that is passed through the object of interest. Other embodiments of imaging device 122 may utilize other imaging techniques, such as, X-ray nano-tomography, focused ion beam scanning electron microscopy, nuclear magnetic resonance, etc.

In some embodiments, the digital image volume may be computationally generated rather than produced by scanning the rock samples 104. In embodiments in which the digital image volume is produced by scanning a rock sample 104, the rock sample 104 may be a naturally occurring rock or a man-made porous material (e.g., a synthetic rock or other porous media).

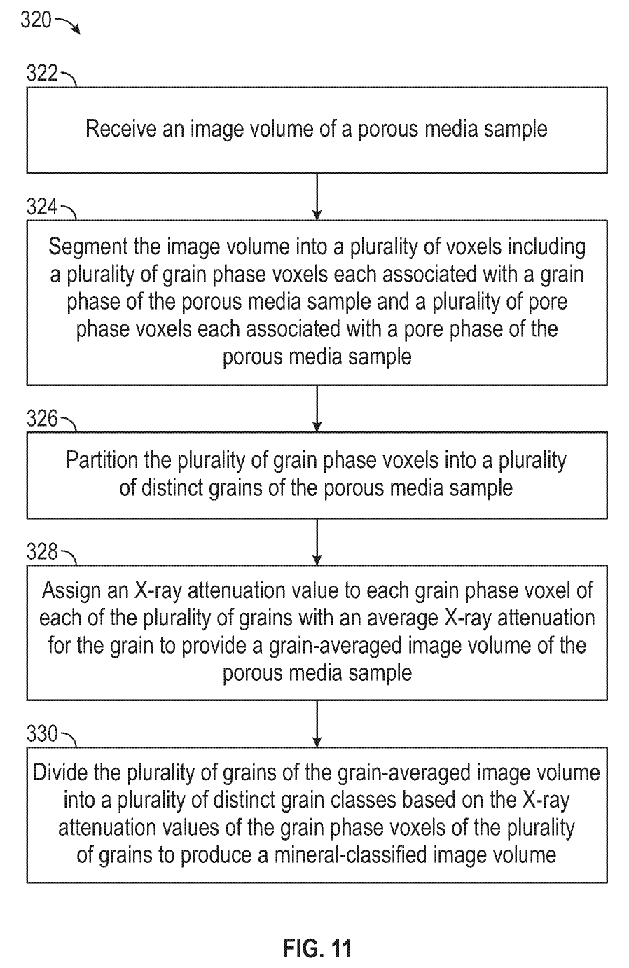

Image volume 128 may be composed of grayscale values representative of the attenuation of the X-ray radiation by the constituents of rock sample 104. In addition, image volume 128 may comprise multiple two-dimensional (2D) slice images stacked along an axis of the rock sample 104, which together form the image volume 128 of rock sample 104. In general, the stacking of the 2D slice images into 3D image volume 128 may be performed by computational resources of imaging device 122 itself, or by a separate computing device 120 from the series of 2D slice images produced by imaging device 122, depending on the architecture of testing system 102. Computing device 120 may comprise or be similar to the computer system 300 shown in FIG. 11.

The images produced by imaging device 122 may be partitioned into a plurality of voxels. In some embodiments, the images produced by the imaging device 122 may be partitioned into non-cubic grid volume elements having varying geometries. The image volume 128 itself may contain different numbers of voxels in the X, Y, and Z directions. Each voxel within a respective 3D digital image has an associated numeric value, or amplitude, that represents the relative material properties of the imaged sample at that location of the medium represented by the digital volume. The range of these numeric values (commonly known as the grayscale range) depends on the type of digital volume, the granularity of the values, the size of the data register (e.g., 8-bit or 16-bit values), and the like. For example, 16-bit data values enable the voxels of an X-ray tomographic image volume to have amplitudes ranging from 0 to 65,536 with a granularity of 1. In some embodiments, testing system 102 may perform image enhancement techniques (e.g., data smoothing, noise reduction) on the 3D digital image. Likewise, other components of testing system 102 (e.g., imaging device 122 itself) may alternatively perform image enhancement in whole or in part. The image volumes 128 captured by the imaging device 122 may comprise the digital images received at block 12 of method 10.

Referring again to FIG. 1, method 10 includes, at block 14, segmenting the image volume into a plurality of discrete phases. In some embodiments, block 14 includes segmenting the voxels of the image volume provided at block 12 into pore phase voxels, grain phase voxels, and MPV phase voxels. In other embodiments, block 14 may instead include segmenting the voxels of the image volume provided at block 12 into only grain phase voxels and pore phase voxels. In still other embodiments, block 14 may include segmenting the image volume into more than three separate phases that may, for instance, segment as a separate phase (e.g., via a thresholding or other suitable algorithm as will be discussed further herein) “bright” inclusions included in the image volume. Such bright inclusions may correspond to different grain phases that are sufficiently distinguishable from one another to be easily partitioned via thresholding and similar techniques.

In some embodiments, the testing system 102 shown in FIG. 2 performs segmentation or other image enhancement techniques on image volume 128 of rock sample 104 to distinguish and label different components or phases of image volume 128 shown in FIG. 3 based on the grayscale values of image volume 128. More specifically, computing device 120 may perform segmentation to identify components, such as pore phase, grain phase, and MPV phase in the image volume 128.

Computing device 120 of FIG. 3 may utilize any of a number of types of segmentation techniques to segment image volume 128. One approach to segmentation is the application of a “thresholding” process to image volume 128, in which computing device 120 chooses a threshold value within the voxel amplitude range. Those voxels having an amplitude below the threshold value are assigned one specific numeric value that denotes pore phase, while those voxels having an amplitude above the threshold are assigned another numeric value that denotes matrix space (i.e., solid material) referred to herein as grain phase. In this approach, thresholding converts a grayscale image volume to a segmented volume of voxels having different numeric values for example, 0, 1, 2 etc., depending on the number of labeled components.

Computing device 120 may utilize other segmentation techniques. For instance, in some embodiments computing device 120 may utilize Otsu's Method in which a histogram-based thresholding technique selects a threshold to minimize the combined variance of the lobes of a bimodal distribution of grayscale values (i.e., the "intra-class variance"). Other examples of automated segmentation techniques of varying complexity may alternatively or additionally be used by computing device 120 to distinguish different features of an image volume, such techniques including Indicator Kriging, Converging Active Contours, Watershedding, and the like.

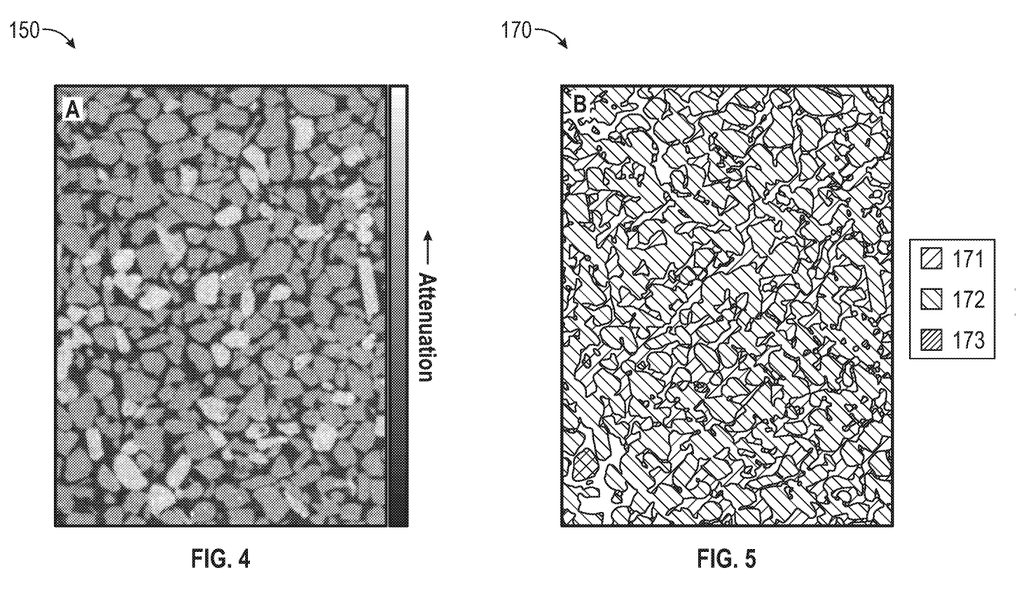

Referring to FIGS. 4 and 5, an exemplary 2D raw or original image slice 150 of an image volume obtained from a porous media sample is shown in FIG. 4. In this exemplary embodiment, raw image slice 150 includes a plurality of pixels each having a brightness or amplitude that is correlated with an attenuation value of the pixel. Particularly, in this exemplary embodiment, pixels having a greater amplitude or brightness have a greater attenuation value while pixels that are darker having a lower amplitude have a lower attenuation value. Raw image slice 150 is generated using single source µCT and thus each pixel has a single attenuation value rather than a set of different attenuation values corresponding to different X-ray energies passed therethrough. The attenuation values for the different pixels of raw image slice 150 may correspond to the density of the material inhabiting the given pixel and where more dense materials are correlated with greater levels of X-ray attenuation. Additionally, raw image slice 150 shown in FIG. 4 may comprise an image slice of an image volume produced at block 12 of method 10.

FIG. 5 illustrates a segmented image slice 170 representing the raw image slice 150 of FIG. 4. once it has been segmented into different distinct phases. For instance, segmented image slice 170 may be the product of applying a thresholding algorithm (e.g., a converging active contours algorithm), Otsu’s Method, and/or other segmentation techniques to the raw image slice 150. In this exemplary embodiment, the different phases labeled or identified in the segmented image slice 170 include pore phase 171, grain phase 172, and MPV phase 173. Alternatively, segmented image slice 170 may label only the pore phase 171 and grain phase 172. Further, segmented image slice 170 may comprise an image slice of a segmented image volume provided at block 14 of method 10.

As an example, partitioning can be performed using any imaging software. During partitioning, a group of voxels can be identified as belonging to a given phase. That is, each voxel or group of voxels define or represent a single phase, and various phases may be represented by different colored (or pattern, shades of grey) voxel or group of voxels as associated to a phase as shown particularly in segmented image slice 170. In some embodiments, segmented image slice 170 is an example of a segmented image volume 128 that partitions the rock sample 104 including structural details of rock sample 104.

Referring again to FIG. 1, at block 16, method 10 includes partitioning the grains of the grain phase voxels identified at block 14. Particularly, block 16 may include distinguishing between the different grains present in the image volume defined by the grain phase voxels thereof. For instance, the different grains of the image volume identified at block 16 may be individually or uniquely labeled to individually each of the different grains identified in the image volume.

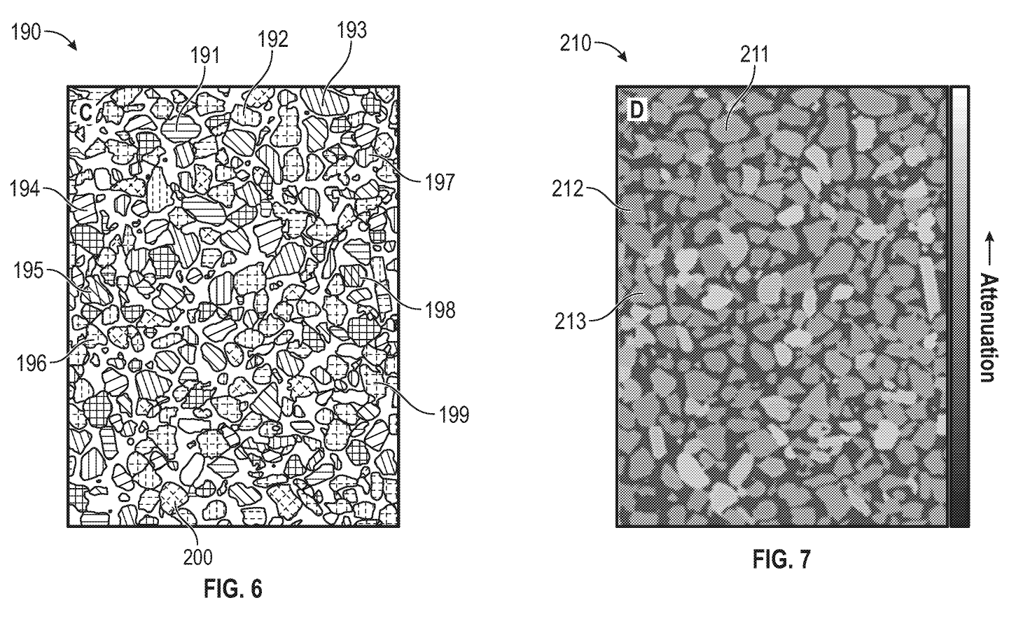

As an example, and referring briefly to FIG. 6, a partitioned image slice 190 from an image volume of a porous medium is shown. In some embodiments, partitioned image slice 190 comprise a partial example of an image volume partitioned at block 16 of method 10 such that individual grains present in the image volume are identified. In this exemplary embodiment, partitioned image slice 190 labels or identifies the individual grains of the grain phase (e.g., partially labeled in FIG. 6 as grains 191-200) contained therein using different shades of color. Alternatively, the different grains of partitioned image slice 190 may be labeled in ways other than color such as a numeric value.

A partitioning algorithm may be applied (e.g., at block 16 of method 10) to the segmented image slice 170 of FIG. 5 to generate the partitioned image slice 190 and thereby delineate different grains in the segmented image slice 170. In certain embodiments, initially a Euclidean Distance Transform (EDT) is applied to the grain phase voxels of the image volume where one or more filters may be applied to the EDT. The output of the EDT may form a topographic map of the partitioning algorithm.

In some embodiments, the partitioning algorithm comprises a watershedding algorithm that treats the segmented image slice 170 as a topographic map where the amplitude (e.g., brightness) of each pixel presents its elevation and where the watershedding algorithm determines or finds “watershed boundaries” that separate catchment basins present in the topographic map. For instance, the amplitudes of different pixels contained in the image volume may represent a topographic surface where low intensity (or inverted high intensity) values form valleys and high intensity values form hills of the topographic map. The watershedding algorithm may treat local minima (e.g., valleys in the topographic map) as starting points (e.g., as "seeds" or markers) for regions that will eventually be flooded with a simulated "water." As water “rises” in the topographic map, different regions within the topographic map fill up until they meet water from an adjacent basin. The lines along which these basins meet are identified as the watershed boundaries. Alternatively, partitioning algorithms other than watershedding algorithms may be used to generate partitioned image slice 190. Similarly, partitioning algorithms other than watershedding algorithms may be implemented at block 16 of the method 10 shown in FIG. 1.

Referring again to FIG. 1, at block 18, method 10 includes averaging the X-ray attenuation for each partitioned grain identified at block 16 whereby a grain-averaged image volume is obtained. Particularly, each grain partitioned at block 16 contains a plurality of unique voxels each having its own attenuation value. In other words, the attenuation values for the voxels of a given partitioned grain may vary substantially from voxel-to-voxel. At block 16, the attenuation values for all of the voxels belonging to each partitioned grain are averaged such that each partitioned grain – rather than having a plurality of different attenuation values that vary voxel-by-voxel – has a single attenuation value corresponding to the average attenuation value for each voxel of the grain. In this manner, each voxel belonging to the partitioned grain is reassigned the average attenuation value for the grain. In other words, following the implementation of block 18, each voxel of the partitioned grain has the same attenuation value corresponding to the average attenuation value for the grain. This process is implemented for each partitioned grain contained in the image volume such that each of the voxels of each partitioned grain have a single attenuation value (e.g., each voxel of a first grain have a first attenuation value, each voxel of a second grain have a second attenuation value, and so on and so forth).

Further, while in this exemplary embodiment block 18 includes averaging the X-ray attenuation values for each partitioned grain at block 16 to “smooth” or reduce the variance in X-ray attenuation values for the grain phase voxels forming the grain, methods other than averaging the attenuation values of the grain phase voxels may be used. In some embodiments, a selected statistical parameter associated with the X-ray attenuation values for each partitioned grain (e.g., a minimum, a maximum, a mode, a mean, a selected percentile) is used to smooth the variance in X-ray attenuation values.

Referring briefly to FIG. 7, a smoothed or grain-averaged image slice 210 from an image volume of a porous medium is shown. In some embodiments, grain-averaged image slice 210 may be obtained via the implementation of block 18 of the method 10 shown in FIG. 1. Particularly, in this example, grain-averaged image slice 210 is based on the partitioned image slice 190 shown in FIG. 6 following the averaging of the attenuation value from image slice 150 across each partitioned grain identified in the partitioned image slice 190. For instance, each pixel of a first grain 211 of grain-averaged image slice 210 has a first brightness (corresponding to a first attenuation value), each pixel of a second grain 212 of grain-averaged image slice 210 has a second brightness (corresponding to a second attenuation value), and each pixel of a third grain 213 of grain-averaged image slice 210 has a third brightness (corresponding to a third attenuation value). In this manner, noise and other imaging artifacts may be minimized when grain-averaging the attenuation values (or otherwise smoothing) the image volume.

Referring again to FIG. 1, at block 20, method 10 includes segmenting the partitioned grains of the grain-averaged image volume obtained at block 18 into a plurality of grain types based on their respective grain-averaged attenuation values to obtain a mineral-classified image volume. Each mineral type may have an attenuation value (e.g., X-ray response) that is unique relative to the other mineral types contained in the mineral-classified image volume. For instance, the total number of partitioned grains of the image volume may be divided into separate mineral types where each mineral type clusters around or towards a unique attenuation value. Thus, a first mineral type of a mineral-classified image volume may each have a grain-averaged attenuation value that clusters around (but does not necessarily equal) an attenuation value for the first mineral type. Similarly, a second mineral type of the mineral-classified image volume may each have a grain-averaged attenuation value that clusters around (but does not necessarily equal) an attenuation value of a second mineral type that varies from the attenuation value of the first mineral type.

In some embodiments, the different attenuation values for each partitioned mineral type correspond to different physical properties (e.g., density) of the grains comprising the different mineral types. In other words, the different mineral types may each identify a different mineral having a unique composition that is present within the image volume. Thus, the number of partitioned mineral types of a given mineral-classified image volume may vary depending on the properties of the porous media sample from which the image volume was originally maintained. In certain embodiments, the attenuation value of a given mineral type about which the grains of the class cluster correspond to a density of the mineral type. In other words, the grains belonging to the mineral type each have a density that clusters around (but does not necessarily equal) the density of the mineral type.

In some embodiments, block 20 includes applying a classification algorithm to the grain phase voxels (and not necessarily other voxels such as the pore phase or MPV phase voxels) of the grain-averaged image volume obtained at block 18 to generate the mineral-classified image volume of the porous media sample. The classification algorithm may divide or separate the different grains of the image volume based on the number of present clusters of attenuation values for the grains of the image volume. In certain embodiments, the classification algorithm applied at block 20 comprises a clustering algorithm such as, for example, a K-means clustering algorithm in which the grains of image volume are grouped into different clusters based on the similarity of their attenuation values (e.g., similarities in their density in some embodiments) without using any predefined labels and without needing to train the algorithm beforehand. In this manner, clustering algorithms may identify distinct clusters of grains which are more similar to each other than to those grains of other clusters.

As an example, K-means clustering includes initially selecting a number of (K) clusters or mineral types. In some embodiments, this selection may be user-defined and may be based on a priori knowledge of the porous media sample such as the number or types of different minerals that are likely to be contained therein in substantial or at least detectable amounts. For instance, this may be based on previous testing of other porous media samples that share features in common with the current porous media sample. In other embodiments, this selection may be automated and performed iteratively until a predefined stabilization threshold has been achieved. For instance, initially two clusters may be selected and the K-means clustering algorithm may be implemented in this manner and an error (or other stabilization threshold) of the final result may be estimated. Subsequently three clusters, four clusters, and so on forth, may be selected and the K-means clustering algorithm implemented for each until the final result sufficiently stabilizes.

In some embodiments, once the number of K clusters have been selected, K initial centroids for each K cluster may be determined either randomly or via one or more different heuristics. Subsequently, distances from each point (e.g., the attenuation value of each grain) and the different K initial centroids are determined and these points are initially assigned to the nearest K initial centroid. Then, for each K cluster, a new centroid is determined by, for instance, taking the mean of all the points assigned to the K cluster. This process may be repeated iteratively until the K centroids stabilize denoting convergence of the clustering algorithm.

K-means clustering is used as an example of a particular clustering algorithm that may be implemented at block 20 of method 10. Alternatively, algorithms other than K-means clustering or clustering algorithms generally may be implemented at block 20. For instance, in some embodiments, the clustering algorithm may include K-medoids clustering, hierarchical clustering, hybrid clustering, fuzzy clustering, and model-based clustering. In some embodiments, a thresholding algorithm may be used instead of a clustering algorithm such as the thresholding algorithms discussed with respect to block 14 of method 10.

Referring again to FIG. 1, at block 22, method 10 includes labeling the MPV phase voxels of the mineral-classified image volume obtained at block 20 to provide a fully-classified image volume that distinguishes the MPV phase voxels from the pore phase voxels and grain phase voxels identified in the mineral-classified image. In some embodiments, block 22 includes applying the previously segmented pore phase and MPV phase voxels with their corresponding pore phase and MPV phase labels.

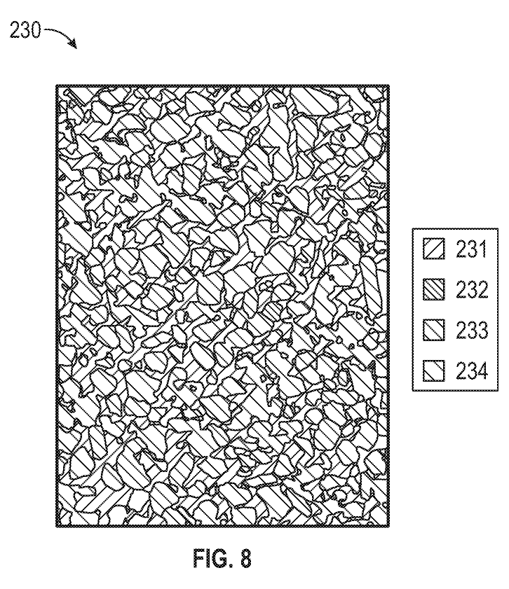

As an example, and referring briefly to FIG. 8, an exemplary fully-classified image slice 230 from an image volume of a porous medium is shown. In some embodiments, the fully-classified image slice 230 is obtained from the grain-averaged image slice 210 shown in FIG. 7 following the application of, for instance, a classification algorithm to the grain-averaged image slice 210. In certain embodiments, a clustering algorithm such as K-means clustering is applied to the grain-averaged image slice 210 to generate the fully-classified image slice 230. Along with pore phase 231 and MPV 232, in this example, the grains of mineral-classified image slice 230 are broken down into a first mineral type 233 and a second mineral type 234 that is different from the first mineral type 233.

In some embodiments, the different mineral types 233 and 234 present in mineral-classified image slice 230 correspond to different minerals present within the porous media sample from which the mineral-classified image slice 230 is obtained. For instance, first mineral type 233 may represent a first mineral and second mineral type 234 may represent a second mineral that is different from the first mineral. Moreover, the grains of each mineral type 233 and 234 may have physical or rock properties that are unique to each other such as, for example, density.

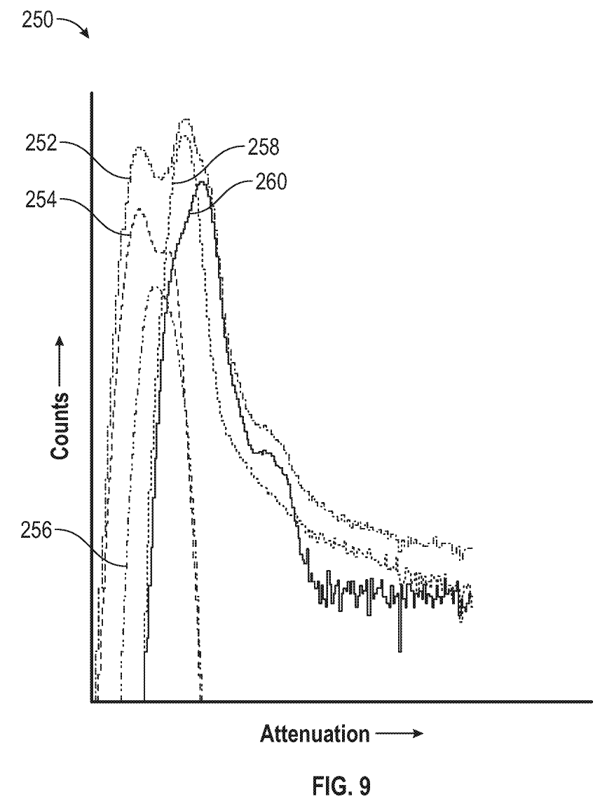

Referring to FIG. 9, a histogram 250 is shown illustrating X-ray attenuation values for each of the voxels contained in an exemplary image volume (e.g., one from which fully-classified image slice 230 is obtained). Particularly, the histogram illustrates the number or “counts” of voxels along a Y-axis thereof and attenuation values along an X-axis thereof for voxels (generally) 252, pore voxels 254, MPV voxels 256, first mineral type 258, and second mineral type 260. Histogram 250 illustrates the divergent clustering of attenuation values for the first mineral type 258 and second mineral type 260 which exhibit distinct peaks having different attenuation values. It may be understood that the significant overlap between the mineral type 258 and mineral type 260 makes it a significant challenge to distinguish between the two when employing only a conventional segmentation algorithm.

In certain embodiments, with the pore phase and MPV phases labeled, and the different mineral types or minerals identified in the image volume, this fully-labeled image volume may be subject to further analysis to yield additional insights into the porous media sample used to obtain the image volume. For example, a priori knowledge of the porous media sample (e.g., knowledge of the subsurface region from which the porous media sample was obtained) may be used to determine or estimate the particular mineral comprising each partitioned mineral type. For instance, knowledge gained from previous experiments conducted with SEM (e.g., QEMSCAN) technology on similar or related porous media samples may be leveraged to estimate the particular minerals comprising the different mineral types of the porous media sample represented by the fully-classified image volume. In some embodiments, upon identifying the specific minerals comprising the different mineral types, the fully-classified image volume may be used to identify different properties or applications of the porous media sample such as relative permeability, reactive flow and transport, and/or geomechanical properties. Such properties may be leveraged in determining the capacity of a subsurface region associated with the porous media sample for producing hydrocarbons and/or other features of interest.

Any of the systems and methods disclosed herein can be carried out (e.g., entirely or partially) on a computer or other device comprising a processor (e.g., a desktop computer, a laptop computer, a tablet, a server, a smartphone, a graphic processing unit (GPU) enabled device or some combination thereof). Referring now to FIG. 10, a computer system 300 suitable for implementing one or more embodiments disclosed herein is shown. The computer system 300 includes a processor 301 (which may comprise one or more central processor units (CPUs) and/or one or more GPUs) that is in communication with memory devices including secondary storage 302, read only memory (ROM) 303, random access memory (RAM) 304, input/output (I/O) devices 305, and network connectivity devices 306.

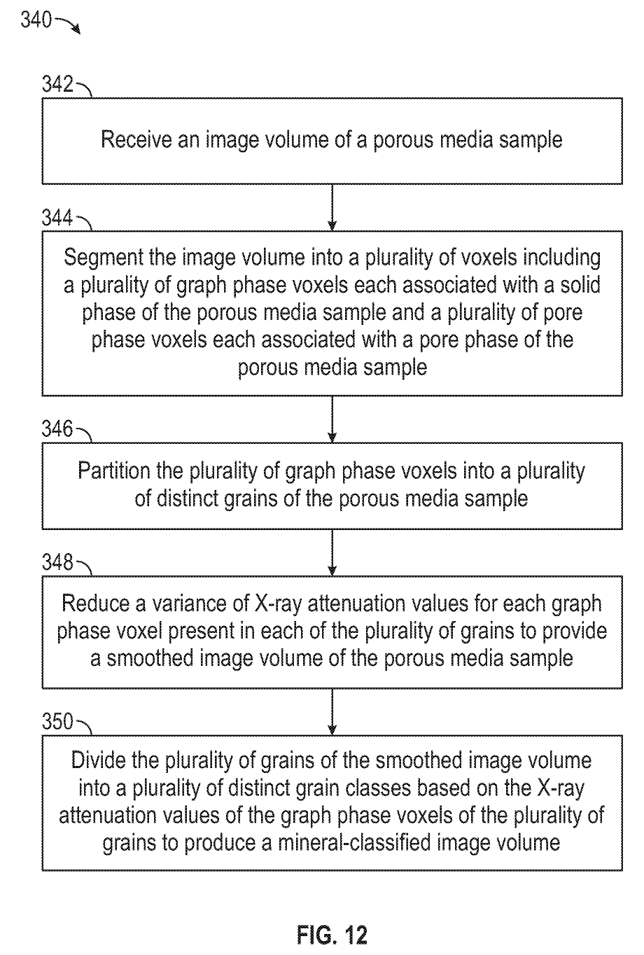

It is understood that by programming and/or loading executable instructions onto the computer system 300, at least one of the processors 301, the RAM 304, and the ROM 303 are changed, transforming the computer system 300 in part into a particular machine or apparatus having the novel functionality taught by the present disclosure. Thus, the RAM 304 and/or the ROM 303 may comprise a non-transitory machine-readable (or computer-readable) medium that may include instructions (which may be referred to herein as machine-readable instructions) that are executable by processor 301 to provide functionality to computer system 300. Thus, in some embodiments, a machine-readable instructions stored on a memory may be executed on a processor, to configure the processor to carry out some or all of the features of the methods described herein (e.g., methods 320 and 340 shown in FIGS. 11 and 12 as will be discussed further herein).

It is fundamental to the electrical engineering and software engineering arts that functionality that can be implemented by loading executable software into a computer can be converted to a hardware implementation by well-known design rules. Decisions between implementing a concept in software versus hardware typically hinge on considerations of stability of the design and numbers of units to be produced rather than any issues involved in translating from the software domain to the hardware domain. Generally, a design that is still subject to frequent change may be preferred to be implemented in software, because re-spinning a hardware implementation is more expensive than re-spinning a software design. Generally, a design that is stable that will be produced in large volume may be preferred to be implemented in hardware (for example in an application specific integrated circuit (ASIC),or field-programmable gate arrays (FPGA)) because for large production runs the hardware implementation may be less expensive than the software implementation. Often a design may be developed and tested in a software form and later transformed, by well-known design rules, to an equivalent hardware implementation in an application specific integrated circuit that hardwires the instructions of the software. In the same manner as a machine controlled by a new ASIC is a particular machine or apparatus, likewise a computer that has been programmed and/or loaded with executable instructions may be viewed as a particular machine or apparatus.

Additionally, after the system 300 is turned on or booted, processor 301 may execute a computer program or application. For example, processor 301 may execute software or firmware stored in ROM 303 or stored in RAM 304. In some cases, on boot and/or when the application is initiated, processor 301 may copy the application or portions of the application from the secondary storage 302 to the RAM 304 or to memory space within processor 301 itself, and processor 301 may then execute instructions of which the application is comprised. In some cases, processor 301 may copy the application or portions of the application from memory accessed via the network connectivity devices 306 or via the I/O devices 305 to the RAM 304 or to memory space within processor 301, and processor 301 may then execute instructions of which the application is comprised. During execution, an application may load instructions into processor 301, for example load some of the instructions of the application into a cache of processor 301. In some contexts, an application that is executed may be said to configure processor 301 to do something, e.g., to configure processor 301 to perform the function or functions promoted by the subject application. When processor 301 is configured in this way by the application, processor 301 becomes a specific purpose computer or a specific purpose machine.

The secondary storage 302 is typically comprised of one or more disk drives or tape drives and is used for non-volatile storage of data and as an over-flow data storage device if RAM 304 is not large enough to hold all working data. Secondary storage 302 may be used to store programs which are loaded into RAM 304 when such programs are selected for execution. The ROM 303 is used to store instructions and perhaps data which are read during program execution. ROM 303 is a non-volatile memory device which typically has a small memory capacity relative to the larger memory capacity of secondary storage 302. The RAM 304 is used to store volatile data and perhaps to store instructions. Access to both ROM 303 and RAM 304 is typically faster than to secondary storage 302. The secondary storage 302, the RAM 304, and/or the ROM 303 may be referred to in some contexts as computer readable storage media and/or non-transitory computer readable media.

I/O devices 305 may include printers, video monitors, electronic displays (e.g., liquid crystal displays (LCDs), plasma displays, organic light emitting diode displays (OLED), touch sensitive displays, etc.), keyboards, keypads, switches, dials, mice, track balls, voice recognizers, card readers, paper tape readers, or other well-known input devices.

The network connectivity devices 306 may take the form of modems, modem banks, Ethernet cards, Omni-Path Architecture (OPA), InfiniBand (IB), universal serial bus (USB) interface cards, serial interfaces, token ring cards, fiber distributed data interface (FDDI) cards, wireless local area network (WLAN) cards, radio transceiver cards that promote radio communications using protocols such as code division multiple access (CDMA), global system for mobile communications (GSM), long-term evolution (LTE), worldwide interoperability for microwave access (WiMAX), near field communications (NFC), radio frequency identity (RFID), and/or other air interface protocol radio transceiver cards, and other well-known network devices. These network connectivity devices 306 may enable processor 301 to communicate with the Internet or one or more intranets. With such a network connection, it is contemplated that processor 301 might receive information from the network, or might output information to the network (e.g., to an event database) in the course of performing the methods described herein. Such information, which is often represented as a sequence of instructions to be executed using processor 301, may be received from and outputted to the network, for example, in the form of a computer data signal embodied in a carrier wave.

Such information, which may include data or instructions to be executed using processor 301 for example, may be received from and outputted to the network, for example, in the form of a computer data baseband signal or signal embodied in a carrier wave. The baseband signal or signal embedded in the carrier wave, or other types of signals currently used or hereafter developed, may be generated according to several known methods. The baseband signal and/or signal embedded in the carrier wave may be referred to in some contexts as a transitory signal.

Processor 301 executes instructions, codes, computer programs, scripts which it accesses from hard disk, floppy disk, optical disk, solid state drives (SSD) (these various disk-based systems may all be considered secondary storage 302), flash drive, ROM 303, RAM 304, or the network connectivity devices 306. While only one processor 301 is shown, multiple processors may be present. Thus, while instructions may be discussed as executed by a processor, the instructions may be executed simultaneously, serially, or otherwise executed by one or multiple processors. Instructions, codes, computer programs, scripts, and/or data that may be accessed from the secondary storage 302, for example, hard drives, floppy disks, optical disks, and/or other device, the ROM 303, and/or the RAM 304 may be referred to in some contexts as non-transitory instructions and/or non-transitory information.

In an embodiment, the computer system 300 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. Further, the computer system 300 may comprise a graphics processing unit (GPU) for processing parallel tasks such as rendering graphics or performing complex computations efficiently. In an embodiment, virtualization software may be employed by the computer system 300 to provide the functionality of a number of servers that is not directly bound to the number of computers in the computer system 300. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or may be hired on an as-needed basis from a third-party provider. Some cloud computing environments may comprise cloud computing resources owned and operated by the enterprise as well as cloud computing resources hired and/or leased from a third-party provider.

In an embodiment, some or all of the functionality disclosed above may be provided as a computer program product. The computer program product may comprise one or more computer readable storage medium having computer usable program code embodied therein to implement the functionality disclosed above. The computer program product may comprise data structures, executable instructions, and other computer usable program code. The computer program product may be embodied in removable computer storage media and/or non-removable computer storage media. The removable computer readable storage medium may comprise, without limitation, a paper tape, a magnetic tape, magnetic disk, an optical disk, a solid-state memory chip, analog magnetic tape, compact disk read only memory (CD-ROM) disks, floppy disks, jump drives, digital cards, multimedia cards, and others. The computer program product may be suitable for loading, by the computer system 300, at least portions of the contents of the computer program product to the secondary storage 302, to the ROM 303, to the RAM 304, and/or to other non-volatile memory and volatile memory of the computer system 300. Processor 301 may process the executable instructions and/or data structures in part by directly accessing the computer program product, for example by reading from a CD-ROM disk inserted into a disk drive peripheral of the computer system 300. Alternatively, processor 301 may process the executable instructions and/or data structures by remotely accessing the computer program product, for example by downloading the executable instructions and/or data structures from a remote server through the network connectivity devices 306. The computer program product may comprise instructions that promote the loading and/or copying of data, data structures, files, and/or executable instructions to the secondary storage 302, to the ROM 303, to the RAM 304, and/or to other non-volatile memory and volatile memory of the computer system 300.

In some contexts, the secondary storage 302, the ROM 303, and the RAM 304 may be referred to as a non-transitory computer readable medium or a computer readable storage media. A dynamic RAM embodiment of the RAM 304, likewise, may be referred to as a non-transitory computer readable medium in that while the dynamic RAM receives electrical power and is operated in accordance with its design, for example during a period of time during which the computer system 300 is turned on and operational, the dynamic RAM stores information that is written to it. Similarly, processor 301 may comprise an internal RAM, an internal ROM, a cache memory, and/or other internal non-transitory storage blocks, sections, or components that may be referred to in some contexts as non-transitory computer readable media or computer readable storage media.

Referring to FIG. 11, an embodiment of a method 320 for mineral mapping an image volume representing a porous media sample (e.g., rock sample 104 shown in FIG. 3) is shown. In some embodiments, method 320 is implemented by a computer system such as, for example, the computer system 300 shown in FIG. 10. Initially, at block 322, method 320 includes receiving an image volume of a porous media sample.

At block 324, method 320 includes segmenting the image volume (e.g., image volume 128 shown in FIG. 3) into a plurality of voxels including a plurality of grain phase voxels each associated with a grain phase of the porous media sample and a plurality of pore phase voxels each associated with a pore phase of the porous media sample. At block 326, method 320 includes partitioning the plurality of grain phase voxels into a plurality of distinct grains (e.g., grains 191-200 shown in FIG. 6) of the porous media sample.

At block 328, method 320 includes assigning an X-ray attenuation value to each voxel of each of the plurality of grains with an average X-ray attenuation for the grain to provide a grain-averaged image volume (e.g., image volume 210 shown in FIG. 7) of the porous media sample. At block 330, method 320 includes dividing the plurality of grains of the grain-averaged image volume into a plurality of distinct mineral types based on the X-ray attenuation values of the solid voxels of the plurality of grains to produce a mineral-classified image volume (e.g. the image volume from which image slice 230 shown in FIG. 8 is obtained).

Referring to FIG. 12, another embodiment of a method 340 for mineral mapping an image volume representing a porous media sample (e.g., rock sample 104 shown in FIG. 3) is shown. In some embodiments, method 340 is implemented by a computer system such as, for example, the computer system 300 shown in FIG. 10. Initially, at block 342, method 340 includes receiving an image volume of a porous media sample.

At block 344, method 340 includes segmenting the image volume (e.g., image volume 128 shown in FIG. 3) into a plurality of voxels including a plurality of solid voxels each associated with a solid phase of the porous media sample and a plurality of pore phase voxels each associated with a pore phase of the porous media sample.

At block 346, method 340 includes partitioning the plurality of solid voxels into a plurality of distinct grains (e.g., grains 191-200 shown in FIG. 6) of the porous media sample.

At block 348, method 340 includes reducing a variance of X-ray attenuation values for each solid voxel present in each of the plurality of grains to provide a smoothed image volume (e.g., image volume 210 shown in FIG. 7) of the porous media sample. At block 350, method 340 includes dividing the plurality of grains of the smoothed image volume into a plurality of distinct mineral types based on the X-ray attenuation values of the solid voxels of the plurality of grains to produce a mineral-classified image volume (e.g., image volume 230 shown in FIG. 8).

The discussion above is directed to various exemplary embodiments. However, one of ordinary skill in the art will understand that the examples disclosed herein have broad application, and that the discussion of any embodiment is meant only to be exemplary of that embodiment, and not intended to suggest that the scope of the disclosure, including the claims, is limited to that embodiment.

The drawing figures are not necessarily to scale. Certain features and components herein may be shown exaggerated in scale or in somewhat schematic form and some details of conventional elements may not be shown in interest of clarity and conciseness.

In the discussion above and in the claims, the terms “including” and “comprising” are used in an open-ended fashion, and thus should be interpreted to mean “including, but not limited to… .” Also, the term “couple” or “couples” is intended to mean either an indirect or direct connection. Thus, if a first device couples to a second device, that connection may be through a direct connection of the two devices, or through an indirect connection that is established via other devices, components, nodes, and connections. In addition, when used herein (including in the claims), the words “about,” “generally,” “substantially,” “approximately,” and the like mean within a range of plus or minus 10%.

While exemplary embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

What is claimed is:

1. A computer-implemented method for mineral mapping an image volume representing a porous media sample, the method comprising:
(a) receiving an image volume of a porous media sample;
(b) segmenting the image volume into a plurality of voxels including a plurality of solid voxels each associated with a solid phase of the porous media sample and a plurality of pore phase voxels each associated with a pore phase of the porous media sample;
(c) partitioning the plurality of solid voxels into a plurality of distinct grains of the porous media sample;
(d) assigning an X-ray attenuation value to each solid voxel of each of the plurality of grains with an average X-ray attenuation for the grain to provide a grain-averaged image volume of the porous media sample; and
(e) dividing the plurality of grains of the grain-averaged image volume into a plurality of distinct mineral types based on the X-ray attenuation values of the solid voxels of the plurality of grains to produce a mineral-classified image volume.

2. The method of claim 1, further comprising:
(f) generating by a single source micro computed tomography (µCT) scanner the image volume of the porous media sample.

3. The method of claim 1, wherein (b) comprises segmenting the image volume into the plurality of voxels including the plurality of solid voxels, the plurality of pore phase voxels, and a plurality of micro-porous volume (MPV) voxels.

4. The method of claim 1, wherein (e) comprises applying a clustering algorithm to the grain-averaged image volume to identify a plurality of discrete clusters in attenuation value for the plurality of grains.

5. The method of claim 4, wherein the clustering algorithm comprises a K-means clustering algorithm.

6. The method of claim 4, further comprising:
(f) receiving a user-defined initial number of clusters for the plurality of mineral types.

7. The method of claim 1, wherein (c) comprises applying a watershedding algorithm to the image volume.

8. The method of claim 1, further comprising:
(f) correlating each of the plurality of mineral types with a mineral corresponding to the mineral type to estimate a composition of the porous media sample.

9. The method of claim 8, wherein the estimated composition of the porous media sample is based on a known composition of an analogous porous media sample.

10. The method of claim 8, further comprising:
(g) determining one or more rock properties or one or more transport properties of the porous media sample using the mineral-classified image volume and the estimated composition of the porous media sample.

11. A computer-implemented method for mineral mapping an image volume representing a porous media sample, the method comprising:
(a) receiving an image volume of a porous media sample;
(b) segmenting the image volume into a plurality of voxels including a plurality of solid voxels each associated with a solid phase of the porous media sample and a plurality of pore phase voxels each associated with a pore phase of the porous media sample;
(c) partitioning the plurality of solid voxels into a plurality of distinct grains of the porous media sample;
(d) reducing a variance of X-ray attenuation values for each solid voxel present in each of the plurality of grains to provide a smoothed image volume of the porous media sample; and
(e) dividing the plurality of grains of the smoothed image volume into a plurality of distinct mineral types based on the X-ray attenuation values of the solid voxels of the plurality of grains to produce a mineral-classified image volume.

12. The method of claim 11, further comprising:
(f) generating by a single source micro computed tomography (µCT) scanner the image volume of the porous media sample.

13. The method of claim 11, wherein (d) comprises replacing original X-ray attenuation values for each solid voxel present in each of the plurality of grains based on a statistical parameter of the original X-ray values of the solid voxels of the grain.

14. The method of claim 13, wherein the statistical parameter comprises an average X-ray attenuation value for the solid voxels of the grain.

15. The method of claim 11, wherein (c) comprises applying a watershedding algorithm to the image volume.

16. The method of claim 11, further comprising:
(f) correlating each of the plurality of mineral types with a mineral corresponding to the mineral type to estimate a composition of the porous media sample.

17. The method of claim 16, wherein the estimated composition of the porous media sample is based on a known composition of an analogous porous media sample.

18. The method of claim 16, further comprising:
(g) determining one or more rock properties or one or more transport properties of the porous media sample using the mineral-classified image volume and the estimated composition of the porous media sample.

19. A system for mineral mapping an image volume representing a porous media sample, the system comprising:
a processor; and
a memory coupled to the processor, wherein machine-readable instructions are stored in the memory, and wherein the machine-readable instructions, when executed on the processor, configure the processor to:
(a) receive an image volume of a porous media sample;
(b) segment the image volume into a plurality of voxels including a plurality of solid voxels each associated with a solid phase of the porous media sample and a plurality of pore phase voxels each associated with a pore phase of the porous media sample;
(c) partition the plurality of solid voxels into a plurality of distinct grains of the porous media sample;
(d) assign an X-ray attenuation value to each solid voxel of each of the plurality of grains with an average X-ray attenuation for the grain to provide a grain-averaged image volume of the porous media sample; and
(e) divide the plurality of grains of the grain-averaged image volume into a plurality of distinct mineral types based on the X-ray attenuation values of the solid voxels of the plurality of grains to produce a mineral-classified image volume.

20. The system of claim 19, wherein the machine-readable instructions, when executed on the processor, configure the processor to:
(f) correlate each of the plurality of mineral types with a mineral corresponding to the mineral type to estimate a composition of the porous media sample.
